(19) United States
(12) Patent Application Publication
(10) Pub. No.: US 2024/0133896 A1
(43) Pub. Date: Apr. 25, 2024

(54) RAPID PROCESSING AND DIRECT TESTING OF SALIVA BIOMARKERS

(71) Applicant: Accure Health Inc., Cambridge, MA (US)

(72) Inventors: Liyun SANG, Cambridge, MA (US); Qiang LIU, Cambridge, MA (US); Victoria SA MOMYER, Cambridge, MA (US)

(73) Assignee: Accure Health Inc., Cambridge, MA (US)

(21) Appl. No.: 18/394,169

(22) Filed: Dec. 22, 2023

Related U.S. Application Data
(63) Continuation of application No. PCT/US2022/034775, filed on Jun. 23, 2022.
(60) Provisional application No. 63/215,276, filed on Jun. 25, 2021.

(51) Int. Cl. G01N 33/68

(52) U.S. Cl. CPC G01N 33/6863; G01N 33/6854; G01N 2333/52

(57) ABSTRACT

The present disclosure relates generally to methods and chemical compositions for saliva sample processing and testing, and more specifically, rapid analysis of saliva biomarkers for detecting and monitoring disease.

### CROSS-REFERENCE TO RELATED APPLICATIONS

This application is a continuation under 35 U.S.C. § 111(a) of PCT International Patent Application No. PCT/US2022/034775, filed Jun. 23, 2022, designating the United States and published in English, which claims priority to and the benefit of U.S. Provisional Application No. 63/215,276, filed Jun. 25, 2021, the entire contents of each of which are incorporated by reference herein.

### FIELD

The present disclosure relates generally to methods and chemical compositions for saliva sample processing and testing, and more specifically, rapid analysis of saliva biomarkers for detecting and monitoring disease.

### BACKGROUND

Saliva has increasingly become an attractive body fluid for diagnostic or prognostic testing, largely due to its ease of collection, non-invasiveness, and affordability, when compared to blood and other types of diagnostic testing. Despite the progress, there are a few factors that have been impeding the wider adoption of saliva testing in clinical settings. One often cited criticism of using saliva as a diagnostic fluid is that biomarkers are present in amounts that are too low to be detected reliably. Another major concern is the lack of a standardized collection procedure. Saliva samples are often heterogeneous and the mucus in saliva affects the performance of saliva-based assays. Particularly, the sensitivity and consistency of rapid saliva tests were relatively low. There is an urgent need to develop standardized saliva processing and testing methods that can be easily implemented in the laboratory and at the point of care.

### SUMMARY

As described herein, the disclosure features saliva treatment compositions and saliva homogenization procedures for saliva sample processing and testing. Also described herein are methods for rapid analysis of saliva biomarkers for detecting and monitoring disease.

In one aspect, provided herein is a composition for saliva treatment, comprising: a saliva sample obtained from a subject; and a mixture of a first non-ionic detergent and a second non-ionic detergent, wherein the mixture is present in an amount sufficient to inactivate at least some or substantially all viruses present in the saliva sample.

In some embodiments, the first and second non-ionic detergents are selected from Triton™ X-100, Triton™ X-114, Nonidet P-40 (NP-40), Igepal® CA-630, n-dodecyl-β-D-maltoside (DDM), Digitonin, Tween™-20 and Tween™-80, glyco-lithocholate amphiphiles (GLC-1, GLC-2, and GLC-3) and glyco-diosgenin amphiphile (GDN).

In some embodiments, the first non-ionic detergent is Triton™ X-100. In some embodiments, the Triton™ X-100 is present in the composition at a concentration of from about 0.1% to about 1%, from about 0.2% to about 0.9%, from about 0.3% to about 0.8%, from about 0.4% to about 0.7%, from about 0.5% to about 0.6%, from about 0.15% to about 0.5%, about 0.15%, about 0.25%, or about 0.5%.

In some embodiments, the second non-ionic detergent is Polysorbate 20. In some embodiments, the Polysorbate 20 is present in the composition at a concentration of from about 0.05% to about 1%, from about 0.1% to about 0.9%, from about 0.2% to about 0.8%, from about 0.3% to about 0.7%, from about 0.4% to about 0.6%, from about 0.15% to about 0.5%, about 0.15%, about 0.25%, or about 0.5%.

In some embodiments, the composition is substantially homogeneous. Homogeneity can be achieved by e.g., mechanical admixing.

Also provided herein is a method of analyzing a saliva sample, comprising: providing the composition disclosed herein; optionally diluting the composition with a diluant; and subjecting the composition to an assay for determining the presence or amount of an analyte of interest.

In some embodiments, the assay is selected from integrated magneto electronic sensing (iMES), enzyme-linked immunosorbent assay (ELISA), flow cytometry, and lateral flow immunoassay. In some embodiments, the assay comprises iMES.

In some embodiments, the analyte of interest is selected from exosomes and/or cytokines. In some embodiments, the analyte of interest is selected from SARS-Cov 2 antigen and/or anti-SARS-Cov 2 IgG antibody.

A further aspect relates to a method of preparing a saliva sample, comprising: providing a saliva sample obtained from a subject; and admixing the saliva sample with a mixture of a first non-ionic detergent and a second non-ionic detergent, wherein the mixture is present in an amount sufficient to inactivate at least some or substantially all viruses present in the saliva sample.

In some embodiments, the admixing step comprises mixing by hand. In some embodiments, the admixing step comprises mixing by mechanical means such as vortexing and centrifuging.

In some embodiments, the first and second non-ionic detergents are selected from Triton™ X-100, Triton™ X-114, Nonidet P-40 (NP-40), Igepal® CA-630, n-dodecyl-β-D-maltoside (DDM), Digitonin, Tween™ -20 and Tween™ -80, glyco-lithocholate amphiphiles

(GLC-1, GLC-2, and GLC-3) and glyco-diosgenin amphiphile (GDN).

In some embodiments, the first non-ionic detergent is Triton™ X-100. In some embodiments, the Triton™ X-100 is present in the composition at a concentration of from about 0.1% to about 1%, from about 0.2% to about 0.9%, from about 0.3% to about 0.8%, from about 0.4% to about 0.7%, from about 0.5% to about 0.6%, from about 0.15% to about 0.5%, about 0.15%, about 0.25%, or about 0.5%.

In some embodiments, the second non-ionic detergent is Polysorbate 20. In some embodiments, the Polysorbate 20 is present in the composition at a concentration of from about 0.05% to about 1%, from about 0.1% to about 0.9%, from about 0.2% to about 0.8%, from about 0.3% to about 0.7%, from about 0.4% to about 0.6%, from about 0.15% to about 0.5%, about 0.15%, about 0.25%, or about 0.5%.

Also provided herein is the admixed saliva sample prepared according to the method disclosed herein.

The details of one or more embodiments are set forth in the accompanying drawings and the description below. Other features and advantages will be apparent from the description and drawings, and from the claims.

### BRIEF DESCRIPTION OF THE DRAWINGS

FIGS. 1A-1D illustrate the relative I_{CD63} signals (FIG. 1A) and the background noise (FIG. 1B-1D) for CD63 expression in extracellular vesicles present in saliva samples, assayed after saliva samples were treated with SPM or other chemical reagents. In FIG. 1A-1C, the signals were obtained after saliva samples went through mechanical mixing procedures. In FIG. 1D, the signals were obtained after saliva samples went through manual mixing by hand only.

FIG. 2 is a graph showing the relative anti-SARS-Cov 2 Spike IgG antibody levels present in saliva samples, assayed after saliva samples were treated with SPM or other chemical reagents.

FIG. 3 is a graph showing the relative SARS-Cov 2 Spike antigen levels present in three saliva samples, assayed after saliva samples were treated with SPM.

FIGS. 4A and 4B illustrate concurrent analyses of SARS-Cov 2 Spike antigen and anti-SARS-Cov 2 Spike IgG in saliva samples, after saliva samples were treated with SPM. FIG. 4A is a graph showing the relative SARS-Cov 2 Spike antigen levels present in saliva samples. FIG. 4B is a graph showing the relative anti-SARS-Cov 2 Spike IgG antibody levels present in saliva samples from study subjects. A cutoff level was selected and shown in dotted lines. Boxes and horizontal bars denote interquartile range (IQR) and median value, respectively. Whisker endpoints are equal to the maximum and minimum values below or above the median±1.5 times the IQR.

FIGS. 5A-5C show the TiMES-Now assay schematic and TiMES automation system. FIG. 5A shows IgG antibodies against the SARS-Cov-2 RBD protein were captured on magnetic particles directly from saliva and labeled with secondary antibodies for electronic detection. FIG. 5B shows that TiMES is a compact, connected automation system controlled wirelessly by a mobile tablet. Up to 8 tests can be performed in parallel and completed in 30 min. FIG. 5C displays the test report automatically generated by the mobile APP immediately after a test is done. Both the measured neutralizing antibody level and the estimated immune protection efficacy are provided in the report.

FIGS. 6A-6B. TiMES-Now enables SARS-CoV-2 nAb monitoring from convalescent and vaccinated patients. Saliva samples were collected from participants who were neither infected nor vaccinated (n=45), and from convalescent participants (FIG. 6A) who were previously tested positive for COVID-19 with a PCR test and not yet vaccinated (n=10), and vaccinated participants (FIG. 6B) who were previously immune naive and at least 15 days post vaccination (n=67). ROC curves were generated and the areas under the curve (AUC) were calculated. A cutoff level was selected and shown in dotted lines. Boxes and horizontal bars denote interquartile range (IQR) and median value, respectively. Whisker endpoints are equal to the maximum and minimum values below or above the median±1.5 times the IQR. The data are presented on a logarithmic scale.

FIG. 7 showcases the longitudinal monitoring of SARS-Cov-2 RBD IgG responses in saliva. Sample were tested with the TiMES-Now SARS-Cov-2 saliva nAb assay and the TiMES readings were plotted and connected with a dotted line. TiMES-now was able to detect positive SARS-CoV-2 nAb from saliva one week post-vaccination. The nAb levels showed steady increases after the first vaccine dose, and peaked about two weeks after the second dose. The data are presented in the logarithmic scale.

FIGS. 8A-8C show the longitudinal and dose-dependent analysis of SARS-Cov-2 RBD IgG responses in immunocompromised patients. FIG. 8A, at the indicated time points after vaccination, saliva samples were collected from immunocompromised (ImmuCM) individuals and healthy participants (healthy). Sample were tested with the TiMES-Now SARS-Cov-2 saliva nAb assay and the TiMES readings were plotted. FIG. 8B shows that the overall SARS-Cov-2 nAb levels were much higher after study participants received both vaccine doses. FIG. 8C shows that even after the second vaccine dose, the levels of nAbs from immunocompromised patients (ImmuCM-2) were still lower than the healthy controls (Healthy-2) (p<0.002). Boxes and horizontal bars denote interquartile range (IQR) and median value, respectively. Whisker endpoints are equal to the maximum and minimum values below or above the median±1.5 times the IQR. The predictive thresholds for reaching 50% and 90% protection efficacy are also indicated. The data are presented in the arithmetic (FIG. 8A) and the logarithmic (FIG. 8B-8C) scales.

FIGS. 9A-9B show that the TiMES-Now saliva assay can monitor immune protection against viral variants. FIG. 9A shows the correlation of RBD IgG responses in serum and saliva. Five matched serum and saliva sample pairs were tested using the TiMES-Now SARS-Cov-2 assay for correlations in levels of anti-Spike RBD IgG, including IgG to the wild-type viral antigen and two of the viral variants. The results showed high correlation between matched serum and saliva samples (R2=0.934). FIG. 9B shows that the IgG responses toward additional viral variants (B.1.617, aka India variant) were tested using saliva from 4 study participants, including an immunocompromised patient S4. The predictive thresholds for reaching 50% and 90% protection efficacy are indicated. The predicted protection against the B.1.351 and B1.617 variants were much lower, especially for subject S4.

FIG. 10 shows the TiMES-Now SARS-Cov-2 nAb assay standard curve. Varying concentrations of anti-SARSCov-2 Spike IgG antibodies were diluted into a negative saliva sample (0.2 ng/mL˜2000 ng/mL) and assayed using the TiMES automation system. The TiMES readings were plotted and a standard curve was generated. The data are presented on a logarithmic scale. The test achieved a large dynamic range (4 orders of magnitude), and a high correlation (R2=0.998) in the analytical range found in clinical samples.

FIGS. 11A-11C demonstrate that the TiMES-Now SARS-Cov-2 nAb saliva assay achieved highly-reproducible results. FIG. 11A shows non-stimulated saliva samples that were self-collected from one study participant. Aliquots of saliva samples were prepared and tested within 24hrs of collection. There was high consistency between aliquot results. FIG. 11B shows three independent saliva samples that were collected from one study participant over a period of 24 hrs (evening, morning, afternoon) and tested. There was only small variation (CV<7%) among test results. FIG. 11C shows the case where a small amount of coffee or toothpaste solution were spiked into the saliva aliquots and compared with control aliquots diluted with water. Coffee and toothpaste may affect test results, therefore study participants were asked to rinse their mouth with water prior to the saliva self-collection. All measurements in (a) and (c) were performed in duplicates, and the data are displayed as mean±SD. AU: arbitrary units from the TiMES readings.

FIGS. 12A-12B show that the TiMES-Now saliva assay allows longitudinal monitoring of SARS-Cov-2 IgG responses against multiple viral variants. Saliva sample were tested with the TiMES-Now SARS-Cov-2 nAb assay and the readings were plotted and connected with a dotted line. TiMES-Now was able to simultaneously monitor immune responses against the WT and the B.1.617.2 variant, prior to and after the 1st, 2nd and 3rd (booster) dose of a COVID-19 mRNA vaccine. FIG. 12A shows the temporal responses of a study subject who never had a COVID-19 infection. FIG. 12B shows the temporal responses of another study subject who was previously infected with SARS-Cov-2 prior to receiving the 1st dose of a COVID-19 mRNA vaccine. In both FIG. 12A and FIG. 12B, the protection against WT and variant was significantly enhanced by the 3rd (booster) dose.

### DETAILED DESCRIPTION

The disclosure features compositions and methods that are useful for saliva sample processing and testing, and more specifically, rapid and consistent analysis of saliva biomarkers for detecting and monitoring disease or immunity.

In one aspect, disclosed herein is a saliva processing matrix (SPM) and related method for saliva-based rapid testing. The SPM and method can allow quantitative measurements of both Covid antigen and antibody in one saliva sample. It can be used for analysis of additional saliva-based biomarkers, including but not limited to extracellular biomarkers and cytokines. The SPM contains specific composition of detergents which can help inactivate the virus and also improve sensitivity and consistency of the saliva test results. Detergents (e.g. Triton™ X-100) have been used previously in the inactivation of viruses. However, to date combining different types of non-ironic detergents to treat saliva has not been reported. It has been demonstrated herein that specific composition of detergents led to surprisingly reliable saliva assay performance and improved both the sensitivity and consistency of the testing results. When the concentrations of detergents in saliva are above the working concentration range, the analyte of interest would lose functionality. Under working concentrations, conventional treatment with a single detergent was not sufficient to achieve reliable saliva assay performance either. The SPM and method disclosed herein can be applied to a variety of saliva assays, including but not limited to integrated magneto electronic sensing (iMES), enzyme-linked immunosorbent assay (ELISA), flow cytometry, and lateral flow immunoassay, both in the laboratory settings and at the point of care.

### Definitions

Unless defined otherwise, all technical and scientific terms used herein have the same meaning as commonly understood by those of ordinary skill in the art to which this disclosure pertains. The following references provide one of skill with a general definition of many of the terms used in this disclosure: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd.

(2002); and A Dictionary of Biology (Oxford Paperback Reference), Martin and Hine (Eds.), Oxford University Press (4^{th} ed., 2000). Further clarifications of some of these terms as they apply specifically to this disclosure are provided herein.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “non-ionic detergents” refer to detergents that have uncharged hydrophilic head groups. They are considered mild surfactants as they break protein-lipid and lipid-lipid associations, but typically not protein-protein interactions, and generally, do not denature proteins. Non-ionic detergents include but are not limited to the Triton™ family (Triton™ X-100, Triton™ X-114, Nonidet P-40 (NP-40), Igepal® CA-630), n-dodecyl-β-D-maltoside (DDM), Digitonin, the Tween™ family (Tween™-20 and Tween™-80), glyco-lithocholate amphiphiles (GLC-1, GLC-2, and GLC-3) and glyco-diosgenin amphiphile (GDN).

The term “Polysorbate 20” refers to a polysorbate-type nonionic surfactant formed by the ethoxylation of sorbitan before the addition of lauric acid. Common commercial brand names include Scattics, Alkest TW 20 and Tween 20. The CAS number of “Polysorbate 20” is 9005-64-5.

The term “Triton™ X-100” refers to (C14H22O(C2H4O)n), which is a nonionic surfactant that has a hydrophilic polyethylene oxide chain (on average it has 9.5 ethylene oxide units) and an aromatic hydrocarbon lipophilic or hydrophobic group. The hydrocarbon group is a 4-(1,1,3,3-tetramethylbutyl)-phenyl group. The CAS number of “Triton™ X-100” is 9002-93-1.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “antigen” herein is used in the broadest sense and encompasses various molecules or molecular structures, such as may be present on the outside of a pathogen, that can be bound by an antigen-specific antibody or B-cell antigen receptor.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

“SARS-Cov-2” refers to a novel coronavirus now called severe acute respiratory syndrome coronavirus 2 (SARS-CoV-2; formerly called 2019-nCoV), including its variants of interest and variants of concern, as defined by the World Health Organization (WHO).

“Covid-19” refers to an infectious disease caused by a novel coronavirus now called severe acute respiratory syndrome coronavirus 2 (SARS-CoV-2; formerly called 2019-nCoV).

“Neurological disorders” refer to disorders that affect the brain as well as the nerves found throughout the human body and the spinal cord. Structural, biochemical or electrical abnormalities in the brain, spinal cord or other nerves can result in a range of symptoms.

By “marker” is meant any protein or polynucleotide having an alteration in expression level or activity that is associated with a disease or disorder.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

Any compositions or methods provided herein can be combined with one or more of any of the other compositions and methods provided herein.

### Saliva Collection Device (SAD)

Saliva collection device is a device used for collection and storage of saliva samples. In some embodiments of the present disclosure, the saliva collection device is a polypropylene tube.

### Saliva Processing Matrix (SPM)

Saliva processing matrix (SPM) is a combination of chemicals used to mix with saliva sample prior to downstream analysis. In some embodiments of the present disclosure, the SPM is a chemical mixture comprising different detergents. In some embodiments, the SPM contains Triton™ X-100 and another detergent. In some embodiments, both detergents are non-ionic detergents. In some embodiments, the Triton™ X-100 is present in the composition at a final working concentration of from about 0.1% to about 1%, from about 0.2% to about 0.9%, from about 0.3% to about 0.8%, from about 0.4% to about 0.7%, from about 0.5% to about 0.6%, from about 0.15% to about 0.5%, about 0.15%, about 0.25%, or about 0.5%. In some embodiments, the SPM contains Triton™ X-100 and Polysorbate 20. In some embodiments, the Polysorbate 20 is present in the composition at a final working concentration of from about 0.05% to about 1%, from about 0.1% to about 0.9%, from about 0.2% to about 0.8%, from about 0.3% to about 0.7%, from about 0.4% to about 0.6%, from about 0.15% to about 0.5%, about 0.15%, about 0.25%, or about 0.5%. In some embodiments, the SPM is added to the SAD prior to saliva sample collection. In some embodiments, the SPM is added to the saliva after collection.

### Saliva Collection Procedure

Unstimulated whole saliva is collected from a subject without any stimulation. In some embodiments, the subject's head is slightly tilted forward to allow saliva to collect on the floor of the mouth and dribble into a saliva collection device.

### Saliva Homogenization Procedure

Saliva homogenization procedure facilitates homogenization of the saliva prior to downstream analysis. In some embodiments, saliva homogenization procedure includes manually inverting or agitating the sample tube to mix saliva with SPM. In some embodiments of the present disclosure, saliva homogenization procedure includes using mechanical mixing instruments (e.g., micro centrifuge, vortex mixer) to mix saliva with SPM. In some embodiments, saliva homogenization procedure includes using a vortex mixer. In some embodiments, the vortexing time ranges from 3 s to 300 s. In some embodiments, saliva homogenization procedure includes using a desktop micro centrifuge to separate clear saliva from debris. In some embodiments, the centrifugation speed ranges from 500 g to 2000 g. In some embodiments, the centrifugation time ranges from 5 s to 10 min.

In some embodiments, following saliva homogenization the saliva SPM mixture can be further diluted with a dilutant. The diluent and extent of dilution can be selected according to the downstream assay. In some embodiments, the dilutant contains PBS. In some embodiments, the dilutant is a blocking buffer containing any of bovine serum albumin (BSA), non-fat dry milk (NFDM), fish gelatin, whole sera, or polyethylene glycol (PEG).

### iMES

Addressing the unmet clinical need for biomarker analysis, integrated Magneto-Electronic Sensing (iMES) technology was recently developed. iMES technology combines biomarker isolation and detection in a single platform and offers distinct advantages: i) disease biomarkers can be analyzed directly from complex media without the need for extensive filtration or centrifugation; ii) the assay can achieve high detection sensitivity through magnetic enrichment and signal amplification; and iii) based on the electronic detection scheme, sensors can be miniaturized and expanded for parallel measurements. In some embodiments, an analyte detection device is used to detect markers in saliva. In some embodiments, the analyte detection device is an integrated magnetic-electrochemical sensing (iMES) system or device, such as those described in PCT International Application No. PCT/US2017/015433, the contents of which are herein incorporated by reference in their entirety.

### TIMES

TiMES (Technology integrated Magneto-Electronic Sensing) is an automation system implementing the iMES technology, such as those described in PCT International Application No. PCT/US2019/054906, the contents of which are herein incorporated by reference in their entirety. TIMES is a next generation system advanced from earlier prototypes of the iMES device. The TIMES system features i) automated assay process; ii) 8× parallel detections; and iii) real-time data analyses and secure cloud integration. Robotic design, electronics, and firmware were optimized to achieve “sample-in-answer-out” workflow automation. TIMES has a small footprint (˜10″×7″×6″; 26 cm×18 cm×15 cm) and can be easily set up in an office or home setting. Proprietary test cartridges and mobile TIMES APP were also customized for conducting specific tests. Up to 8 tests can be performed in parallel and completed in 30 min. Once the assays are completed, the mobile TIMES APP conducts automatic analyses and provides individual test reports in real time.

### Extracellular Vesicles (EVs)

Extracellular vesicles are membrane-bound phospholipid vesicles that are produced and secreted or otherwise released by cells, such as cells in healthy and unhealthy (e.g., cancerous) tissues. In some embodiments, the extracellular vesicles are exosomes. Extracellular vesicles can comprise cellular constituents (e.g., proteins, nucleic acids, and the like) from their originating cells, which can be used diagnostically. Combined with their relatively large abundance and ubiquitous presence in bodily fluids (e.g., urine, blood, ascites, saliva, CSF), extracellular vesicles offer significant advantages for disease diagnosis, treatment, and management.

In some embodiments of the present disclosure, extracellular vesicles are derived from or present in saliva obtained from a subject (e.g., a mammal, such as a human). A subject may not have or be suspected of having a disease or condition. In some embodiments, a subject has or is suspected of having a disease or condition, such as cancer, Covid-19, or neurological disorders.

### EV Marker Analysis in Saliva

In some embodiments, the presence of EV marker in saliva can be analyzed using the iMES technology, post treatment with SPM.

For instance, immune markers can be released to saliva and associated with extracellular vesicles (EVs). Treatment with SPM can improve immune marker detection using the iMES technology. Magnetic beads conjugated to antibodies specific to EV markers can capture EVs from a SPM treated saliva sample. Subsequently, the sample can bind to secondary molecules specific to immune marker (e.g., a labeling ligand, such as biotinylated antibodies specific against immune marker), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the immune marker expressing EVs have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of immune marker expressing EVs. In turn, this current can be measured by an MCU or other computing device, and the resulting information can be used for investigative, or predictive purposes. In some cases, an instrument can render analyses automatically or semi-automatically based on the measurements.

Although the detection of EV-associated immune marker in saliva using the iMES technology is described above, this is merely an example. In practice, immune marker can be either free-floating (e.g., free-floating in plasma, urine, or any other biological sample) or expressed on the surface or inside of a biological structure (e.g., extracellular vesicle). The disease can include but not limited to Covid-19, other infectious diseases, cancer and neurological disorders. The detection technology can include but not limited to ELISA, flow cytometry, and lateral flow immunoassay.

### Antibody Analysis in Saliva

In some embodiments, the presence and amount of anti-SARS-Cov 2 IgG antibody in saliva can be detected using the iMES technology, post treatment with SPM.

For instance, anti-SARS-Cov 2 IgG antibody can be released to saliva. Treatment with SPM can improve antibody detection using the iMES technology. Magnetic beads conjugated to protein fragment of SARS-Cov 2 can capture anti-SARS-Cov 2 IgG from a SPM treated saliva sample. Subsequently, the sample can be treated with secondary molecules binding to IgG (e.g., an oxidizing enzyme conjugated to an antibody recognizing IgG). The sample can then be combined with an electron mediator solution (e.g., a solution containing 3,3′,5,5′-tetramethylbenzidine, TMB).

The sample can be subsequently analyzed using the iMES sensing systems described herein. As the anti-SARS-Cov 2 IgG antibodies have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of anti-SARS-Cov 2 IgG antibodies. In turn, this current can be measured by an MCU or other computing device, and the resulting information can be used for investigative, diagnostic or surveillance purposes.

In some cases, the output of a potentiostat can be compared to a threshold or reference level, and the presence or absence of immunity in a patient can be detected based on the comparison. For example, if the output of a potentiostat is sufficiently high (e.g., a current that exceeds a reference or threshold level), a diagnosis regarding the presence of immunity can be rendered. However, if the output of the potentiostat is relatively low (e.g., a current that does not exceed the reference or threshold level), a diagnosis regarding the absence of immunity may be rendered. In some cases, an instrument can render analyses automatically or semi-automatically based on the measurements.

Although the detection of anti-SARS-Cov 2 IgG antibody in saliva is described above, this is merely an example. In practice, the antibody can include but not limited to monoclonal antibodies, polyclonal antibodies, multispecific antibodies (e.g., bispecific antibodies), and antibody fragments so long as they exhibit the desired antigen-binding activity. The disease can include but not limited to Covid-19, other infectious diseases, or cancer. The detection technology can include but not limited to ELISA, flow cytometry, and lateral flow immunoassay.

### Antigen Detection in Saliva

In some embodiments, the presence and amount of SARS-Cov 2 antigen in saliva can be detected using the iMES technology, post treatment with SPM.

For instance, SARS-Cov 2 can be present in saliva. Treatment with SPM can improve viral antigen detection using the iMES technology. Magnetic beads conjugated to antibodies specific to SARS-Cov 2 Spike (S) or Nucleoprotein (N) can capture SARS-Cov 2 antigen from a SPM treated saliva sample. Subsequently, the sample can bind to secondary molecules specific to SARS-Cov 2 antigen protein (e.g., a labeling ligand, such as biotinylated antibodies specific against immune marker), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the SARS-Cov 2 viral antigen have been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of SARS-Cov 2 antigen. In turn, this current can be measured by an MCU or other computing device, and the resulting information can be used for investigative, diagnostic or surveillance purposes.

In some cases, the output of a potentiostat can be compared to a threshold or reference level, and the presence or absence of viral infection in a patient can be detected based on the comparison. For example, if the output of a potentiostat is sufficiently high (e.g., a current that exceeds a reference or threshold level), a diagnosis regarding the presence of infection can be rendered. However, if the output of the potentiostat is relatively low (e.g., a current that does not exceed the reference or threshold level), a diagnosis regarding the absence of infection may be rendered. In some cases, an instrument can render analyses automatically or semi-automatically based on the measurements.

Although the detection of SARS-Cov 2 antigen in saliva is described above, this is merely an example. In practice, the antigen can be associated with other types of diseases, including but not limited to infectious diseases, cancer and neurological disorders. The detection technology can include but not limited to ELISA, flow cytometry, and lateral flow immunoassay.

### Cytokine Analysis in Saliva

In some embodiments, the presence and amount of cytokine in saliva can be detected using the iMES technology, post treatment with SPM.

For instance, cytokines can be present in saliva. Treatment with SPM can improve cytokine detection using the iMES technology. Magnetic beads conjugated to antibodies specific to cytokine markers (e.g. IL-2, IL-6, IL-10, IL-12, IL-17) can capture cytokine markers from a SPM treated saliva sample. Subsequently, the sample can bind to secondary molecules specific to the cytokine markers (e.g., a labeling ligand, such as biotinylated antibodies specific against immune marker), and treated with tertiary molecule specific to the secondary molecules and having an oxidizing enzyme (e.g., a streptavidin-HRP). The sample can then be combined with an electron mediator solution (e.g., a solution containing 3,3′,5,5′-tetramethylbenzidine, TMB).

The sample can be subsequently analyzed using the iMES sensing systems described herein. As the cytokine protein has been captured by the magnetic beads, they are concentrated near the electrodes of the sensing system. Further, due to the potential induced across the electrodes (e.g., the working electrode and the reference electrode), an oxidation-reduction reaction is induced between the electron mediators and the oxidizing enzyme. As a result, a current is induced across one of the electrodes (e.g., the counter electrode), correlating with the presence and prevalence of the cytokine marker In turn, this current can be measured by an MCU or other computing device, and the resulting information can be used for investigative, diagnostic or surveillance purposes. For example, a relatively high current can correspond to a relatively high concentration of specific cytokine in saliva, and may be a predictor that the patient is more likely to develop a cytokine storm.

In some cases, the output of a potentiostat can be compared to a threshold or reference level, and the presence or absence of cytokine storm in a patient can be detected based on the comparison. For example, if the output of a potentiostat is sufficiently high (e.g., a current that exceeds a reference or threshold level), a prediction regarding the development of a cytokine storm can be rendered. However, if the output of the potentiostat is relatively low (e.g., a current that does not exceed the reference or threshold level), a prediction regarding the absence of cytokine storm may be rendered. In some cases, an instrument can render analyses automatically or semi-automatically based on the measurements.

Although the analyses of cytokine markers in saliva using iMES is described above, this is merely an example. In practice, additional immune markers associated with diseases can be analyzed, including but not limited to infectious diseases, cancer and neurological disorders. The detection technology can include but not limited to ELISA, flow cytometry, and lateral flow immunoassay.

### Methods and Uses

Methods are provided herein for saliva sample processing and testing, and more specifically, rapid analysis of saliva biomarkers for detecting and monitoring disease. In some embodiments, the methods comprise mixing saliva sample with a saliva processing matrix (SPM). In some embodiments, the methods comprise detecting and characterizing one or more markers associated with a disease (e.g., Covid-19, cancer) in a saliva sample. In some embodiments, the saliva is obtained from a subject (e.g., a mammal, such as a human).

In some embodiments, detecting the antigen, antibody, or markers in saliva comprises an immunoassay (e.g., antibody-mediated sequestration or labeling of the one or more markers). In some embodiments, an iMES or analogous system or device is employed to detect the antigen, antibody, or markers present in saliva. For example, a viral antigen, antibody or EV marker may be captured using an affinity capture agent capable of binding or otherwise interacting with the antibodies, or the membrane of a virus, or an extracellular vesicle. In some embodiments, the extracellular vesicle or virus is effectively captured by an antibody that specifically binds to a marker present in or on the extracellular vesicle or virus. In some embodiments, the extracellular vesicle or virus is permeabilized prior to marker labeling. In some embodiments, the extracellular vesicle or virus is broken open prior to maker labeling.

For example, magnetic beads can be conjugated to antibodies or proteins that specifically bind one or more markers of interest. The magnetic beads are mixed with a saliva sample containing a viral antigen, antibodies or extracellular vesicles, such that these analytes are captured on the bead. The bead can then be isolated from the sample with a magnet. In some embodiments, the sample is treated with a secondary molecule that specifically binds to a marker. In some embodiments, the sample is treated with a tertiary composition comprising a moiety that specifically binds the secondary molecules. In some embodiments, the tertiary composition further comprises a moiety having oxidizing enzymatic activity (e.g., a streptavidin-HRP). The sample can then be combined with an electron mediator solution (e.g., a solution containing 3,3′,5,5′-tetramethylbenzidine, TMB). The voltage or current generated can vary due to the oxidation-reduction reaction. The currents generated are detected using a sensing system, such as those described herein, and the collected data can be used for predictive or diagnostic purposes.

The present disclosure also contemplates monitoring the immunity of a subject previously infected with a virus or vaccinated with a vaccine. Using the methods and compositions described herein, samples obtained from the subject can be tested at different time points. In some embodiments, a first sample is obtained from the patient prior to infection or vaccination. In some embodiments, subsequent samples are obtained after the infection or vaccination. By analyzing antibodies associated with the disease, determinations can be made as to whether immunity is generated, or whether the vaccination is effective. For example, in some cases, an increased amount of the antibody after infection or vaccination is indicative of immunity. Conversely, if the amount of antibodies observed remains low, the infection or vaccination is not effectively inducing immunity. Additionally, if a decreased amount of the antibody is observed, the immunity may be diminished over a period of time.

### Kits

One aspect of the present disclosure provides a kit for saliva sample processing and testing. In some embodiments, the kit comprises a saliva collection device (SAD). In some embodiments, the kit comprises saliva processing matrix (SPM).

In some embodiments, the kit further comprises magnetic beads conjugated to a capture molecule (e.g., an antibody) that specifically binds one or more of the markers described herein or another marker associated with cancer. In some embodiments, the kit comprises a secondary molecule that specifically binds to a ligand on an extracellular vesicle (e.g., a labeling ligand, such as biotinylated antibodies specific against the one or more markers). In some embodiments, the kit further comprises a tertiary composition comprising a moiety that specifically binds the secondary molecules. In some embodiments, the tertiary composition further comprises a moiety having oxidizing enzymatic activity (e.g., a streptavidin-HRP). In some embodiments, the kit comprises an electron mediator solution (e.g., a solution containing 3,3′,5,5′-tetramethylbenzidine, TMB).

In some embodiments, the kit comprises a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding reagents.

The kit may provide instructions for using the kit to collect and process saliva samples. It may also include information about the detection and characterization of markers in saliva associated with specific disease. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization.

The following examples are put forth so as to provide those of ordinary skill in the art with a complete disclosure and description of how to make and use the assay, screening, and therapeutic methods of the disclosure, and are not intended to limit the scope of the disclosure.

### EXAMPLES

### Example 1. Direct Analyses of EV Markers in Saliva

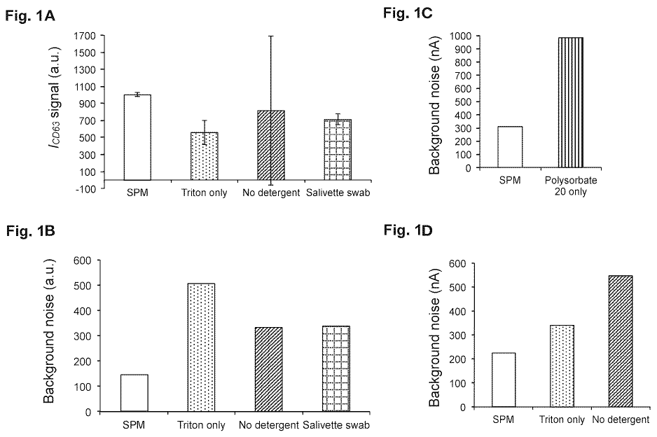

Saliva samples were supplemented with a saliva processing matrix (SPM) and mixed thoroughly by inverting the tube. Each sample was then aliquoted without any purification. Aliquot samples were either used directly, or went through a homogenization procedure, such as mechanical mixing, manual mixing, or centrifugation with a Salivette (SARSTEDT) swab. Each aliquot (40 μL per marker) was then incubated with magnetic beads for EV capture. The bead-bound EVs were consecutively labeled for target markers and HRP, and transferred onto the sensing electrode. EV marker CD63 was measured with iMES along with their respective IgG background noises. We compared impact of SPM and different homogenization procedures on the background noises Ii g G and EV marker net signals I_{CD63} (FIG. 1). The I_{CD63} signals, coefficient of variation (CV) and signal-to-noise ratio (SNR) were also shown in Table 1. SPM treatment resulted in the highest signals, highest consistency (lowest CV) and the highest SNR.

TABLE 1
Comparison of different saliva treatment methods

| Method | Signal (I_{CD63}) | CV | SNR |
|---|---|---|---|
| SPM | 1000 | 2.4% | 6.8 |
| Triton only | 557.4 | 25.6% | 1.1 |
| No detergent | 814.3 | 107.3% | 2.5 |
| Salivette swab | 710.3 | 9.0% | 2.1 |

### Example 2. Direct Analyses of Anti-SARS-Cov 2 IgG Antibody in Saliva

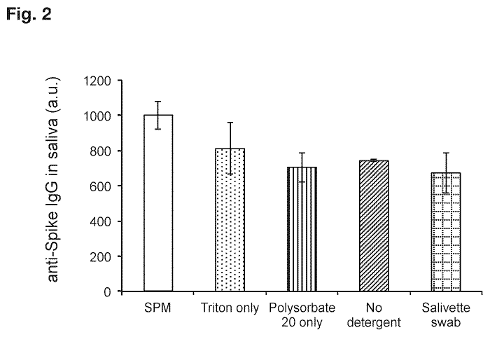

Saliva samples were supplemented with a saliva processing matrix (SPM) and mixed thoroughly by inverting the tube. Each sample was then aliquoted without any purification. Aliquot samples were either used directly, or went through different homogenization procedures. Each aliquot (40 μL per marker) was then incubated with magnetic beads for antibody capture. The bead-bound anti-SARS-Cov 2 Spike IgG were consecutively labeled for anti-human IgG-HRP, and transferred onto the sensing electrode. anti-SARS-Cov 2 Spike IgG was measured with iMES. The signal I Spike IgG was obtained. We compared impact of SPM and different homogenization procedures on the signals I_{Spike IgG} (FIG. 2). The SPM treatment resulted the highest signals with good consistency.

### Example 3. Direct Analyses of SARS-Cov 2 Antigen in Contrived Saliva Samples

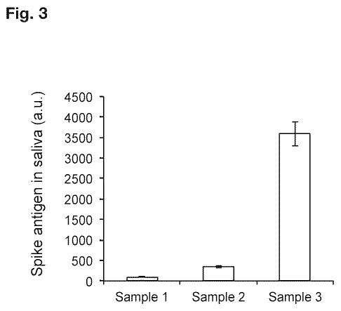

Varying concentrations of SARS-Cov-2 Spike protein (Acro, active trimer) were spiked into control saliva samples. The resulting contrived saliva samples were supplemented with a saliva processing matrix (SPM) and mixed thoroughly by inverting the tube. Each sample was then aliquoted without any purification. Each aliquot (40 μL per marker) was then incubated with magnetic beads for antigen capture. The bead-bound SARS-Cov 2 antigen were consecutively labeled for anti-SARS-Cov2 antibody and HRP, and transferred onto the sensing electrode. SARS-Cov 2 Spike antigen was measured with iMES. The signal I_{Spike} was obtained (FIG. 3). The SPM treatment resulted in good consistency in samples with different concentrations of SARS-Cov-2 Spike antigen.

### Example 4. Concurrent Analyses of SARS-Cov 2 Antigen and Anti-SARS-Cov 2 IgG in Clinical Saliva Samples

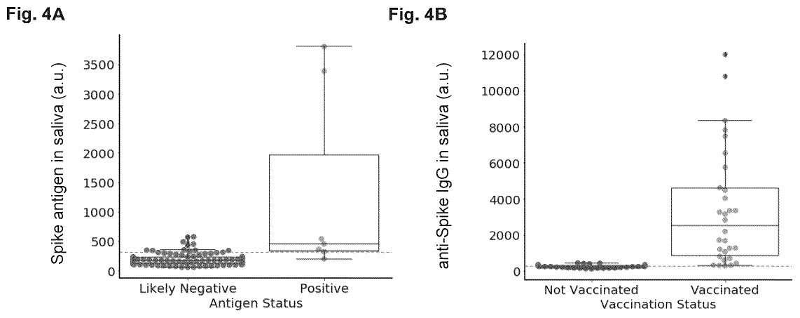

Clinical saliva samples were supplemented with a saliva processing matrix (SPM) and mixed thoroughly by inverting the tube. Each sample was then irradiated using a bench-top ultraviolet laboratory sterilizer (Benchmark Scientific B1450) at an energy level 45mJ/cm2 to inactivate the virus. Each sample was then incubated with specific magnetic beads for antigen or antibody capture. The bead-bound SARS-Cov 2 antigen or anti-SARS-Cov 2 IgG antibody were consecutively labeled for the detection antibody and HRP, and transferred onto the sensing electrode. Signals were measured with iMES. The signal I_{Spike} and I_{Spike IgG} were obtained for each saliva sample (FIG. 4). Compared with samples from study participants who were neither infected nor vaccinated, SPM treatment allowed the detection of SARS-Cov-2 Spike antigen in saliva samples from infected patients (FIG. 4A). In the meantime, SPM treatment also allowed the detection of anti-SARS-CoV-2 IgG in saliva samples from patients that were tested at least 15 days after receiving a COVID-19 vaccine (Pfizer, Moderna, J&J, Sinopharm, Novavax) (FIG. 4B).

### Example 5. A Rapid Saliva Test for Monitoring Immune Protection against SARS-CoV-2 and its Variants

The following example demonstrates that the saliva processing matrix (SPM) and the saliva homogenization procedure can be used to provide a non-invasive, inexpensive and data-driven solution for large-scale immunity surveillance and for predictive modeling of vaccine efficacy.

### INTRODUCTION

The COVID-19 pandemic, caused by infection with severe acute respiratory syndrome coronavirus 2 (SARS-CoV-2), has resulted in significant morbidity and mortality globally. Immunity to SARS-CoV-2 gained either through infection or vaccination has been shown to confer protection against reinfection and/or reduce the risk of clinically severe outcomes. However, the vaccination levels in many countries have not yet reached the threshold estimated to permit sufficient protection at the population level. There is ongoing transmission and emergence of viral variants that may escape control by humoral immune response induced by existing vaccines. For example, whereas the messenger RNA vaccine BNT162b2 (Pfizer—BioNTech) was shown to have an 89.5% efficacy in preventing disease caused by the B.1.1.7 variant, its efficacy against the B.1.351 infection was only 75%. Another challenge arose from the reported reduction in vaccine efficacy in immunocompromised patients (e.g. cancer patients receiving immunotherapy) . It is not clear whether the duration of protective immunity is reduced in the immunocompromised patients or when a booster dose would be needed.

One of the gold standards to study immunity is through the measurements of neutralizing antibodies (nAbs) in serum. Measuring nAb has provided a deeper insight into understanding the vaccine efficacy on various subpopulations. It was also reported that nAb levels are highly predictive of immune protection from symptomatic SARS-CoV-2 infection. Most of the existing SARSCoV-2 nAb assays were conducted in high-complexity labs using venous blood, which requires blood drawing by a trained phlebotomist. The commercially available SARS-CoV-2 antibody tests based on finger-prick blood mainly provide qualitative results and the test accuracy has been under scrutiny. Saliva, on the other hand, may serve as a simple non-invasive alternative to venous blood to monitor humoral immune responses on a large scale following SARS-CoV-2 infection or vaccination. Recent studies have indeed demonstrated that serum levels of IgG against Spike and receptor binding domain (RBD) positively correlate with IgG levels in matched saliva samples from convalescent patients, and also tightly correlates with the neutralizing titer. It is promising that the immune protection against SARS-CoV-2 and its variants can be estimated from a simple saliva test.

We showcase TiMES (technology integrated magneto-electronic sensing), a novel digital technology platform for rapidly monitoring the neutralizing antibody levels against SARS-CoV-2 in human saliva samples. The technology has been previously demonstrated to accurately detect cancer, transplant rejection and sepsis from blood or urine specimens. It outperformed conventional assays in several aspects: the detection sensitivity was >100 fold higher compared to that of ELISA; the assay was fast, inexpensive and can be easily performed at the point of care. With the newly-developed TiMES-Now saliva assay, we were able to rapidly assess SARS-CoV-2 immunity following vaccination, monitor neutralizing antibody levels over time, and model immune protection against major variants. The test also allowed a closer examination on how different subpopulations, particularly immunocompromised patients, responded to the vaccines. TiMES-Now provides an affordable, data-driven and highly-accurate solution for the rapid assessment of personalized immune response as well as population immunity, and to inform vaccination strategy.

### RESULTS

### A Rapid and Automated TiMES-Now Assay for Detecting SARS-CoV-2 Neutralizing Antibodies (nAb) in Saliva

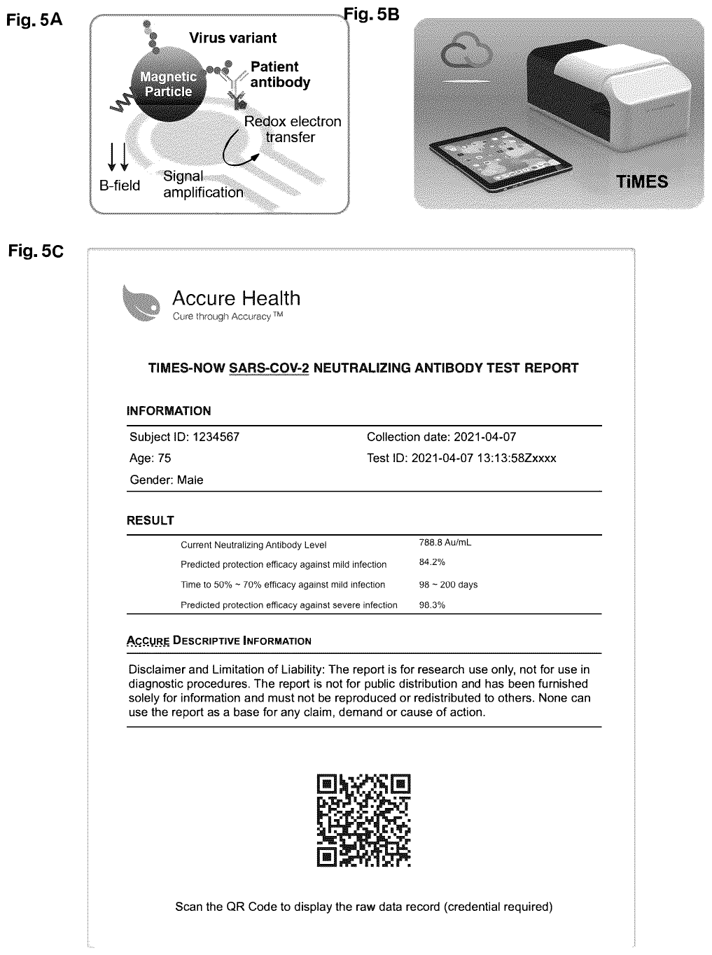

In order to monitor the neutralizing antibody response to SARS-CoV-2, we developed the rapid and automated TiMES-Now nAb assay based on integrated magneto-electronic sensing (iMES). We initially focused on the IgG antibody to the Spike RBD, as neutralizing antibodies are directed to the Spike protein and the RBD domain plays key roles in viral entry. The assay incorporates a dual signal amplification scheme to achieve a superior detection sensitivity to existing rapid antibody tests. The assay first uses magnetic particles (MP) to capture antibodies against the SARS-Cov-2 RBD protein directly from saliva and subsequently labels them with enzyme-linked anti-human IgG antibodies. MP-antibody complexes are then magnetically concentrated on top of a sensing electrode; redox reactions and electron transfer from the electrode generated electrical current as an analytical readout (FIG. 5A). Both magnetic concentration and enzymatic reaction serve to amplify the analytical signal, boosting the detection sensitivity.

To improve throughput and reproducibility of the SARS-Cov-2 nAb assay, the tests were performed using a portable and automated TiMES system. The newly developed TiMES is a next generation system advanced from earlier prototypes of the iMES device. The TiMES system features i) automated assay process; ii) 8× parallel detections; and iii) real-time data analyses and secure cloud integration. Robotic design, electronics, and firmware were optimized to achieve “sample-in-answer-out” workflow automation. TiMES has a small footprint (˜10″×7″×6″; 26 cm×18 cm×15 cm) and can be easily set up in an office or home setting (FIG. 5B). Proprietary test cartridges and mobile TiMES APP were also customized for conducting the TiMES-Now SARS-Cov-2 nAb tests. Up to 8 tests can be performed in parallel and completed in 30 min. Once the assays are completed, the mobile TiMES APP conducts automatic analyses and provides individual test reports in real time (FIG. 5C, Table 2).

TABLE 2
TiMES-Now SARS-Cov-2 nAb Assay summary

| | |
|---|---|
| Validated sample type | Saliva, serum |
| Analyte type | SARS-Cov-2 Spike RBD IgG antibodies, |
| | including variants |
| Reproducibility | CV <5% |
| Dynamic range | 4 orders of magnitude |
| Test throughput | 8 tests/device/30 min (automated test and data |
| | report), hands-on time~5 min |
| Data analysis | Mobile APP for real-time analysis and reporting, |
| | sync with cloud and HIPAA ready |

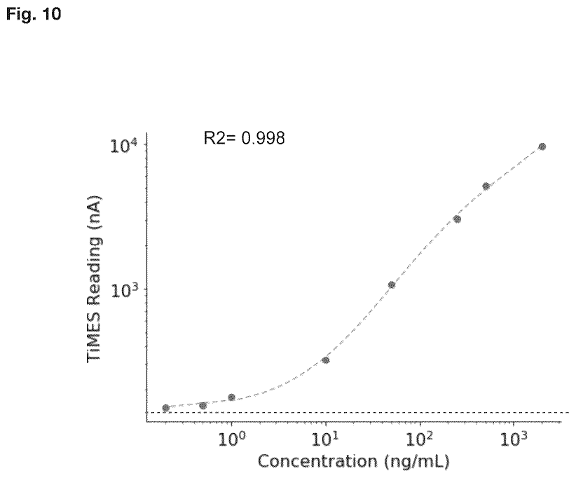

For quantitative analyses, we established a standard curve for each production batch, and integrated the standard curve as a built-in function in the TiMES APP. The range of the standard curve was selected based on physiologically-relevant concentrations of anti-SARS-Cov-2 Spike IgG antibodies found in patient samples. Varying concentrations of anti-SARS-Cov-2 Spike IgG antibodies were spiked into saliva from persons (no history of COVID-19 infection or vaccination) and assayed. The TiMESNow anti-SARS-Cov-2 nAb assay achieved excellent fitting (R2=0.998) in the selected analytical range (FIG. 10).

### TiMES-Now Enables SARS-CoV-2 nAb Monitoring from Convalescent and Vaccinated Patients

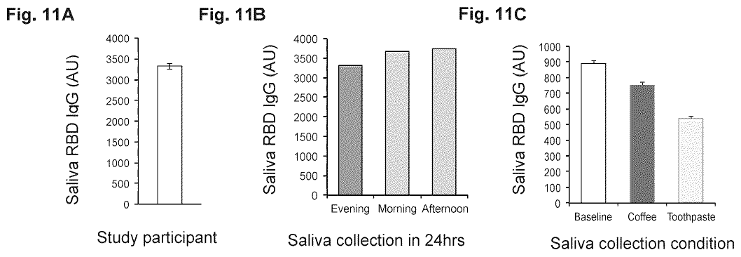

To ensure accurate and reproducible results, we investigated the consistency of the TiMES-Now SARSCoV- 2 nAb assay. Non-stimulated saliva samples were self-collected from study participants who were fully vaccinated (Materials and Methods). Aliquots of saliva samples were prepared, stored at 4° C. and tested within 48 hrs of collection. Coefficient of variation (CV=s.d./mean) was calculated for each set of aliquot measurements. TiMES-Now SARS-CoV-2 nAb assay achieved high reproducibility, with a CV<5% (FIG. 11A). We further studied whether saliva collection time affects the test results. Three independent saliva samples were collected from the same study participant within a 24 hr time frame (evening, morning, afternoon) and tested. TiMES-Now SARS-CoV-2 nAb assay achieved a CV<7%.

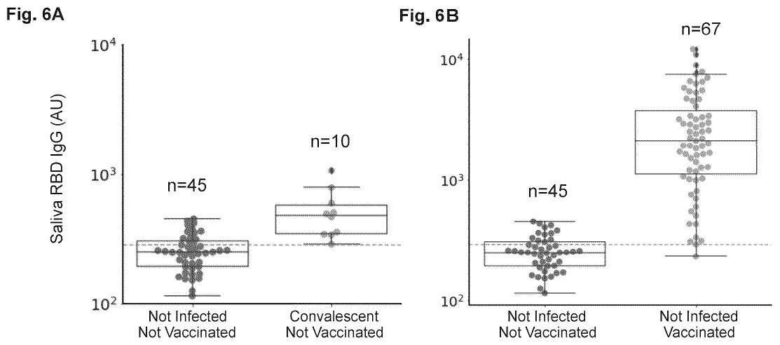

We evaluated the performance of the TiMES-Now SARS-CoV-2 nAb assay using freshly-collected saliva samples from convalescent or vaccinated patients (Table 3). Compared with samples from study participants who were neither infected nor vaccinated (n=45), samples from COVID-19 subjects were mostly positive for SARS-CoV-2 nAb (n=10; AUC=0.92) between 1-9 months post infection (FIG. 6A). The majority of saliva samples from patients that were tested at least 15 days after receiving a COVID-19 vaccine (Pfizer, Moderna, J&J, Sinopharm, Novavax) also showed positive SARS-CoV-2 nAb results (n=67, AUC=0.97) (FIG. 5B).

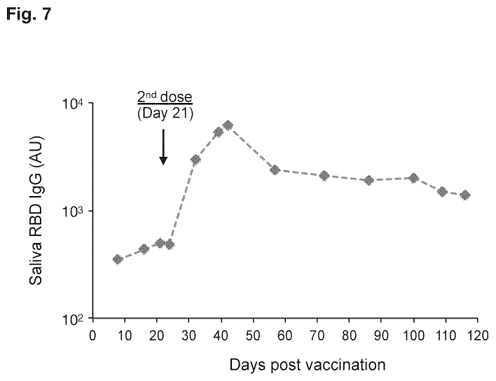

To better understand the temporal evolution of immune response, we conducted longitudinal monitoring of the nAb levels using the TiMES-Now SARS-CoV-2 saliva nAb assay. The test was able to detect positive SARS-CoV-2 nAb from saliva about one week after the participants received the first dose of a COVID vaccine. The saliva nAb levels peaked after the second dose, stayed high over a period of ˜120 days, and then started to decline around 110˜120 days post vaccination (FIG. 7). These results were consistent with the published nAb titters from serum samples.

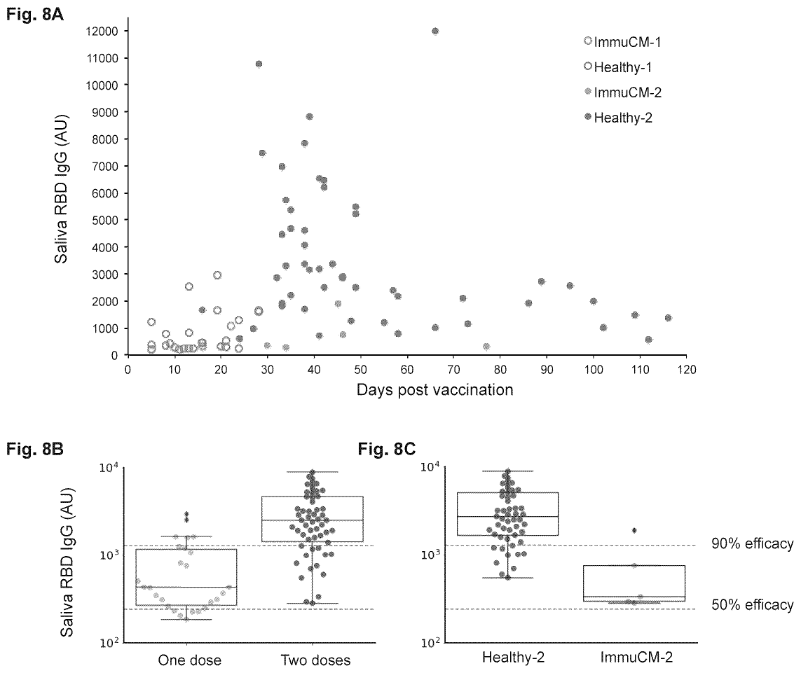

Recent studies suggest that neutralizing antibody levels are highly predictive of immune protection from symptomatic SARS-CoV-2 infection. We thus analyzed the saliva nAb levels from some “high-risk” sub populations and estimated whether there were sufficient nAb for immune protection. Consistent with the recently published studies on vaccine efficacy, we observed that immunocompromised individuals (such as patients taking immunosuppressant drugs or receiving treatments which may compromise the immune system) had relatively lower levels of nAb than healthy participants (FIG. 8A). While the second dose generally provided a boost to the nAb levels, the overall levels of nAbs from immunocompromised patients were still lower than the healthy controls (FIG. 8B-C). We estimated the saliva nAb level for 50% protection against detectable SARS-CoV-2 infection based on a recently published predictive model. At the time of testing, the nAb levels from these immunocompromised patients were still above the 50% efficacy threshold. However, modeling of the nAb decay over the first 120 d after immunization suggests that a significant loss in protection from SARS-CoV-2 infection may occur sooner in these “high-risk” patients.

### TiMES-Now Saliva Assay Monitors Immune Protection Against Viral Variants

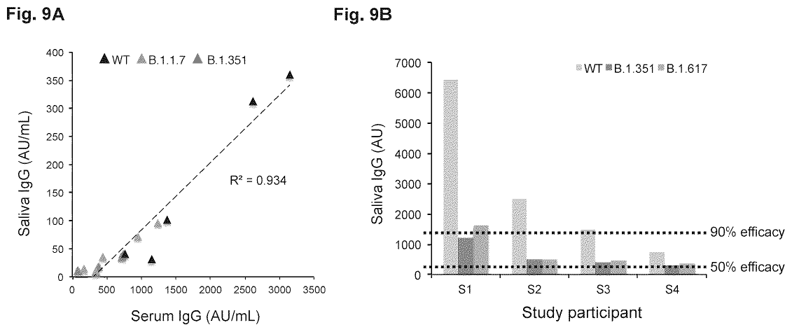

In addition to the declining neutralization titer over time, reduced vaccine efficacy to different viral variants have also been reported. Particularly, it has been shown that the neutralization titer against the B.1.351 variant in vaccinated individuals was significantly lower compared with the early wild-type strain. The gold standard for measuring the neutralization titer is based on venous serum samples. Recent studies, however, have demonstrated the potential use of saliva: IgG against Spike and RBD in the serum positively correlated with matched saliva samples from convalescent patients, and also tightly correlates with the neutralizing titer. We thus applied the TiMES-Now SARS-CoV-2 nAb assay to test whether there was a positive correlation between the saliva nAb levels and the serum nAb levels, to different viral variants. Matched saliva and serum samples from five fully vaccinated patients were tested to measure the levels of nAb to the wild-type Spike RBD as well as to two of the variants of concern: B.1.1.7 and B.1.351. Our results showed a high correlation (R2=0.934) between the saliva nAb levels and the serum nAb levels, including their response to the variants (FIG. 9A). The data also supported a reduction in nAb efficacy against the B.1.351 variant. Taken together, the TiMES-Now SARS-Cov-2 nAb saliva assay demonstrated rapid and accurate assessment of variant response in vaccinated patients.

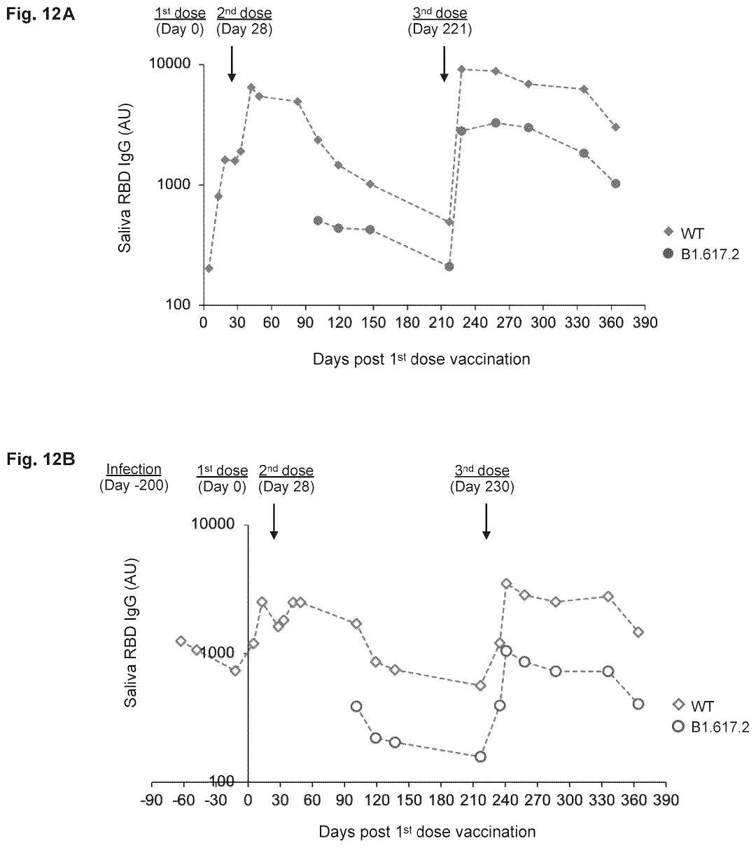

Predictive modeling suggests that a lower neutralization titer is likely to reduce efficacy for immune protection. We therefore examined the nAb levels and estimated the efficacy for immune protection against several variants using the TiMES-Now SARS-Cov-2 saliva nAb assay. We focused on three emerging variants (B.1.351, B1.617, B1.617.2), as they harbor critical mutations which may enhance viral transmissions. While the predicted immune protection against the wild-type (WT) strain was mostly above the 90% efficacy threshold, the predicted protection against these variants were lower, particularly for an immunocompromised patient S4 (FIG. 9B). For people who either had or did not have a COVID-19 infection, three doses of COVID-19 mRNA vaccination enhanced the immune protection against the WT and the viral variants (FIG. 12).

TABLE 3
Patient demographics

| Patient Characteristics | n = 111 [n (%)] |
|---|---|
| Age (mean; range) | 52.7 (18-78) |
| Sex | |
| Female | 104 (94%) |
| Male | 7 (6%) |
| Comorbidities | |
| Asthma | 14 (13%) |
| Hypertension | 15 (14%) |
| Diabetes | 2 (2%) |
| Cancer (prior or current) | 12 (11%) |
| Other⁺ | 3 (3%) |
| Immunocompromised* | |
| Yes, and vaccinated | 7 (6%) |
| Yes, and not vaccinated | 9 (8%) |
| No | 95 (86%) |
| Vaccination Status | |
| Not Vaccinated | 41 (37%) |
| Partially Vaccinated | 15 (13%) |
| Fully Vaccinated | 34 (31%) |
| N/A | 21 (19%) |
| Vaccine Manufacturer | |
| Johnson and Johnson | 3 (6%) |
| Moderna | 11 (23%) |
| Pfizer | 30 (65%) |
| Other^{&} | 3 (6%) |
| Collection Location | |
| Clinic | 105 (95%) |
| Home | 6 (5%) |
| COVID-19 Exposure | |
| Prior infection (confirmed) | 10 (9%) |
| Prior exposure (not confirmed) | 12 (11%) |
| No exposure | 88 (79%) |
| Unknown | 1 (1%) |

*Taking immunosuppressant drugs or receiving treatment that may compromise immune system
• Cystic fibrosis, lung injury, hypertrophic cardiomyopathy
^{&}Sinopharm, Akston Biosciences, Novavax

### Example 6: Materials and Methods

### Saliva Samples

Saliva samples were collected from consented donors under IRB-approved protocols. Samples were collected using a saliva collection device, transported to our laboratory and analyzed. De-identified clinical information (such as age, gender, ethnicity, diagnosis, disease stage, treatment history, etc) were also provided.

### Salivette Swab Treatment of Saliva

Whole saliva is collected from a subject without any stimulation and dribble into a saliva collection device. The unused cotton swab was taken out of a Salivette (SARSTEDT) and transferred to the saliva collection device to absorb the collected saliva sample. The absorbed cotton swab was then transferred back to the Salivette. Salivette was centrifuged for 2 minutes at 1,000×g yields a clear saliva sample in the conical tube, following Salivette vendor's instruction. The saliva recovered was used for downstream analysis.

### Saliva Treatment with SPM

After collection, saliva was mixed with the saliva processing matrix (SPM) briefly. Saliva treatment procedures include shaking the sample tube by hand, or using mechanical mixing instruments (e.g., micro centrifuge, vortex mixer) to mix saliva with SPM.

### Immunomagnetic Beads

Magnetic beads coated with epoxy groups (Dynabeads M-270 Epoxy, Invitrogen) were suspended in 0.1 M sodium phosphate solution at room temperature for 10 min. The magnetic beads were separated from the solution with a permanent magnet and resuspended in the same solution. Antibodies or proteins were added and mixed thoroughly. 3 M ammonium sulfate solution were added, and the whole mixture was incubated overnight at 4° C. with slow tilt rotation. The beads were washed twice with PBS solution and finally resuspended in PBS with 1% bovine serum albumin (BSA).

### Biotinylated Antibodies

Sulfo-NHS-biotin (10 mM, Pierce) solution in PBS was incubated with antibodies for 1 hour at room temperature. Unreacted sulfo-NHS-biotin was removed using Zeba spin desalting column, 7K MWCO (Thermo Scientific). Antibodies were kept at 4° C. until use.

### iMES Assay

After treatment with SPM, 40 μL of saliva was mixed with immunomagnetic bead solution and dilution buffer for a few minutes at room temperature. The magnetic beads were separated from the solution with a permanent magnet, and then re-suspended in buffer with antibodies of interest and oxidizing enzyme, and mixed for a few minutes at room temperature. The magnetic beads were separated, mixed with UltraTMB solution (ThermoFisher Scientific), and loaded on top of the screen-printed electrode. After 5 seconds, chronoamperometry measurement was started with the electrochemical sensor. The current levels in the range of 110-120 seconds were averaged. In some embodiments, an iMES system or device can be used, such as those described in PCT International Application No. PCT/US2017/015433, the contents of which are herein incorporated by reference in their entirety.

### Clinical Study

Study subjects were consented and enrolled in the study with approval from the Biomedical Research Alliance of New York Institutional Review Board (BRANY IRB #A20-08-597-840). Subject recruitment, and saliva collection were conducted at the New England Facial & Cosmetic Surgery Center in Danvers, Massachusetts. Saliva was self-collected at the study site and submitted to the study staff. In cases where the study subject was not able to visit the study site, saliva collection was self administered at home and delivered to the study site. A screening questionnaire was completed by each study subject, and de-identified questionnaire data were collected and analyzed. Immunocompromised individuals were patients taking immunosuppressant drugs or receiving treatments which may compromise the immune system. Non-vaccinated convalescent patients were adults with a prior positive COVID-19 PCR test by self-report who met the definition of recovery by the Centers for Disease Control.

### Sample Collection and Processing in the Clinical Study

Whole saliva was collected from a subject without any stimulation. SPM was pre-loaded into the SAD to help improve sample safety and stability. After saliva collection, sample was mixed thoroughly with the SPM in the SAD. Samples were tested within 48 hrs of collection, or

aliquoted and stored at −80° C. For matched serum and saliva samples, venous blood was collected by standard phlebotomy into a BD serum tube. Serum was separated by centrifugation for 10 minutes at 2000 g and then stored at −20 C. Saliva was collected within 24 hrs of blood collection.

### TiMES-Now SARS-Cov-2 nAb Assay

40 uL of saliva (or 10 uL of serum) was loaded to the TiMES-Now SARS-Cov-2 nAb test cartridge (ISO 13485 certified), inserted into a TiMES automation device. Tests were performed using the TiMES system. Results were generated automatically with the TiMES APP and transferred to a password protected, encrypted cloud database to meet HIPAA compliance. To establish a standard curve, varying concentrations of anti-SARS-Cov-2 Spike IgG antibodies (Sino Biological) were spiked into a negative saliva sample and assayed using the TiMES automation system. A curve was fitted using the TiMES readings and the antibody concentrations. The TiMES system achieved high intra-cartridge and inter cartridge consistency from testing antibody standards. A built-in standard curve was therefore generated for each production batch, saved in the TiMES APP, and automatically calibrated to provide an accurate antibody concentration for each test. For variant studies, viral antigens were obtained from a commercial vendor (Sino Biological) and integrated into the TiMES-Now test cartridges.

### Modeling of the Relationship Between Saliva nAb Level and Immune Protection

To model the protective efficacy using the data from the TiMES-Now SARS-Cov-2 saliva nAb assay, we first established an equation to convert the TiMES-Now SARS-Cov-2 saliva nAb readings into corresponding serum neutralization titer, based on data from the present study and the published clinical trial data. Two assumptions were made: (1) the 50% geometric mean neutralization titer presented in Pfizer's phase 1 trial is equivalent to that in the present study; (2) saliva and serum readings are highly correlated (R2=0.934). Once the TiMES-Now SARS-Cov-2 saliva nAb readings were converted to serum neutralizing titer, we adapted a predictive logistic model to arrive at the protective efficacy against SARS-CoV-2 infection:

$${E}_{I} ( n \vert {n}_{50} , k ) = \frac{1}{1 + {e}^{- k ( n - {n}_{50} )}} ,$$

where E_{I} is the protective efficacy of an individual given the neutralization level n. The parameter n₅₀ is the neutralization level at which an individual will have a 50% protective efficacy (that is, half the chance of being infected compared with an unvaccinated person). The steepness of this relationship between protective efficacy and neutralization level is determined by the parameter k. From the TiMESNow SARS-Cov-2 saliva nAb level, we further estimated the time to reach a certain protective efficacy (50% or 70%) based on the published nAb half-life.

### Other Embodiments

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

What is claimed is:

1. A composition for saliva treatment, comprising:
a saliva sample obtained from a subject; and
a mixture of a first non-ionic detergent and a second non-ionic detergent, wherein the mixture is present in an amount sufficient to inactivate at least some or substantially all viruses present in the saliva sample.

2. The composition of claim 1, wherein the first and second non-ionic detergents are selected from Triton™ X-100, Triton™ X-114, Nonidet P-40 (NP-40), Igepal® CA-630, n-dodecyl-β-D-maltoside (DDM), Digitonin, Tween™-20 and Tween™-80, glyco-lithocholate amphiphiles (GLC-1, GLC-2, and GLC-3) and glyco-diosgenin amphiphile (GDN).

3. The composition of claim 2, wherein the first non-ionic detergent is Triton™ X-100.

4. The composition of claim 3, wherein the Triton™ X-100 is present in the composition at a concentration of from about 0.1% to about 1%, from about 0.2% to about 0.9%, from about 0.3% to about 0.8%, from about 0.4% to about 0.7%, from about 0.5% to about 0.6%, from about 0.15% to about 0.5%, about 0.15%, about 0.25%, or about 0.5%.

5. The composition of claim 1, wherein the second non-ionic detergent is Polysorbate 20.

6. The composition of claim 5, wherein the Polysorbate 20 is present in the composition at a concentration of from about 0.05% to about 1%, from about 0.1% to about 0.9%, from about 0.2% to about 0.8%, from about 0.3% to about 0.7%, from about 0.4% to about 0.6%, from about 0.15% to about 0.5%, about 0.15%, about 0.25%, or about 0.5%.

7. The composition of claim 1, wherein the composition is substantially homogeneous.

8. A method of analyzing a saliva sample, comprising:
providing the composition of claim 1;
optionally diluting the composition with a diluant; and
subjecting the composition to an assay for determining the presence or amount of an analyte of interest.

9. The method of claim 8, wherein the assay is selected from integrated magneto electronic sensing (iMES), enzyme-linked immunosorbent assay (ELISA), flow cytometry, and lateral flow immunoassay.

10. The method of claim 8, wherein the assay comprises iMES.

11. The method of claim 8, wherein the analyte of interest is selected from exosomes and/or cytokines.

12. The method of claim 8, wherein the analyte of interest is selected from SARS-Cov 2 antigen and/or anti-SARS-Cov 2 IgG antibody.

13. A method of preparing a saliva sample, comprising:
providing a saliva sample obtained from a subject; and
admixing the saliva sample with a mixture of a first non-ionic detergent and a second non-ionic detergent, wherein the mixture is present in an amount sufficient to inactivate at least some or substantially all viruses present in the saliva sample.

14. The method of claim 13, wherein the admixing step comprises mixing by hand.

15. The method of claim 13, wherein the admixing step comprises mixing by mechanical means such as vortexing and centrifuging.

16. The method of claim 13, wherein the first and second non-ionic detergents are selected from Triton™ X-100, Triton™ X-114, Nonidet P-40 (NP-40), Igepal® CA-630, n-dodecyl-β-D-maltoside (DDM), Digitonin, Tween™-20 and Tween™-80, glyco-lithocholate amphiphiles (GLC-1, GLC-2, and GLC-3) and glyco-diosgenin amphiphile (GDN).

17. The method of claim 16, wherein the first non-ionic detergent is Triton™ X-100.

18. The method of claim 17, wherein the Triton™ X-100 is present in the composition at a concentration of from about 0.1% to about 1%, from about 0.2% to about 0.9%, from about 0.3% to about 0.8%, from about 0.4% to about 0.7%, from about 0.5% to about 0.6%, from about 0.15% to about 0.5%, about 0.15%, about 0.25%, or about 0.5%.

19. The method of claim 16, wherein the second non-ionic detergent is Polysorbate 20.

20. The method of claim 19, wherein the Polysorbate 20 is present in the composition at a concentration of from about 0.05% to about 1%, from about 0.1% to about 0.9%, from about 0.2% to about 0.8%, from about 0.3% to about 0.7%, from about 0.4% to about 0.6%, from about 0.15% to about 0.5%, about 0.15%, about 0.25%, or about 0.5%.

21. The admixed saliva sample prepared according to the method of claim 13.
